(19) United States
(12) Patent Application Publication
(10) Pub. No.: US 2022/0215537 A1
(43) Pub. Date: Jul. 7, 2022

(54) METHOD FOR IDENTIFYING AND CLASSIFYING PROSTATE LESIONS IN MULTI-PARAMETRIC MAGNETIC RESONANCE IMAGES

(71) Applicant: SOCIEDADE BENEFICENTE ISRAELITA BRASILEIRA HOSPITAL ALBERT EINSTEIN, São Paulo (BR)

(72) Inventors: Silvio MORETO PEREIRA, São Paulo (BR); Victor MARTINS TONSO, São Paulo (BR); Pedro Henrique DE ARAÚJO AMORIM, São Paulo (BR); Ronaldo HUEB BARONI, São Paulo (BR); Heitor DE MORAES SANTOS, São Paulo (BR); Guilherme GOTO ESCUDERO, São Paulo (BR); Artur AUSTREGESILO SCUSSEL, São Paulo (BR)

(21) Appl. No.: 17/609,295

(22) PCT Filed: May 7, 2020
(86) PCT No.: PCT/BR2020/050153

§ 371 Date: Nov. 5, 2021

(30) Foreign Application Priority Data
May 7, 2019 (BR) 10 2019 009339 0

(51) Int. Cl. G06T 7/00; G06T 7/11

(52) U.S. Cl. CPC G06T 7/0012; G06T 7/11; G06T 2207/20084; G06T 2207/30081; G06T 2207/30096; G06T 2207/10088

(57) ABSTRACT

A method for identifying and classifying prostate lesions in multi-parametric magnetic resonance images, which includes a module for zonal segmentation of the prostate, a module for identifying suspected prostate lesion areas, and a module for classifying lesions, which uses T2-weighted image sequences, ADC maps and diffusion-weighted images (DWI) from the multi-parametric magnetic resonance imaging to provide a probability of clinically significant suspected cancerous areas.

### FIELD OF THE INVENTION

The present invention relates to a method for identifying and classifying prostate lesions in multi-parametric magnetic resonance images and, more specifically, to a computer-aided method capable of identifying and classifying prostate lesions according to their malignancy.

### BACKGROUND OF THE INVENTION

Multi-parametric magnetic resonance imaging (mp-MRI) is an imaging method that allows the assessment of prostate disease with high spatial resolution and high soft tissue contrast. Mp-MRI comprises a combination of high-resolution anatomical images with at least one functional imaging technique, such as dynamic contrast enhancement (DCE) and diffusion-weighted imaging (DWI).

Given its characteristics, mp-MRI has become an important tool in the detection and staging of prostate cancer (PC), allowing an increase in the detection of this type of tumor.

One of the methods of detection of PC so far most recommended by urology societies is screening with Prostate Specific Antigen (PSA) dosage and digital rectal examination (DRE). If one or both are altered, a histopathological study of tissue obtained by randomized ultrasound-guided prostate biopsy is performed.

The state of the art already provides for the use of mp-MRI in the clinical practice of urologists prior to biopsy to accurately stratify the chance of finding a clinically significant lesion and guide the biopsy, preferably with an image-guided fusion biopsy procedure.

A challenge present in the application of this type of technique for the identification of PC is the growing demand for properly trained radiologists capable of reading and interpreting the exams.

Thus, automated methods were developed to interpret the results of this type of technique.

Document US2017/0176565, for example, describes methods and systems for the diagnosis of prostate cancer, comprising extracting texture information from MM imaging data for a target organ, and the identification of frequent texture patterns can be indicative of cancer. A classification model is generated based on the determined texture features that are indicative of cancer, and diagnostic cancer prediction information for the target organ is then generated to help diagnose cancer in the organ.

Document US2018/0240233, on the other hand, describes a method and an apparatus for automated detection and classification of prostate tumors in multi-parametric magnetic resonance images (MRI). A set of multi-parametric MRI images of a patient, including a plurality of different types of MRI images, is received. Simultaneous detection and classification of prostate tumors in the multi-parametric MRI image set is performed by using a trained multi-channel image-image convolutional encoder-decoder.

Despite the recent solutions in development, the need for an efficient and low-cost method, capable of quickly and accurately performing the interpretation of mp-MRI results, remains in the state of the art.

### OBJECTIVES OF THE INVENTION

It is an objective of the present invention to provide a method for identifying and classifying prostate lesions in multi-parametric magnetic resonance images that is capable of reading multi-parametric magnetic resonance images, automatically segmenting the anatomy of the prostate and detecting clinically significant areas suspected of prostate cancer.

It is one more of the objectives of the present invention to provide a method for identifying and classifying prostate lesions in multi-parametric magnetic resonance images that allows a more assertive identification of lesions, by previously performing the zonal segmentation of the prostate in transitional and peripheral zones.

It is yet another objective of the present invention to provide a method for identifying and classifying prostate lesions in multi-parametric magnetic resonance images that does not use K-trans maps, eliminating the need for contrast ingestion by the patient during the resonance procedure and reducing risks to the patient and costs associated with the procedure.

### BRIEF DESCRIPTION OF THE INVENTION

The present invention achieves these and other objectives through a method for identifying and classifying prostate lesions in multi-parametric magnetic resonance images, comprising a module for zonal segmentation of the prostate, a module for identifying suspected prostate lesion areas, and a module for classifying lesions.

Thus, the method comprises executing the module for zonal segmentation of the prostate comprising an algorithm to segment, from T2-weighted image sequences of multi-parametric magnetic resonance images, the prostate peripheral and transitional zones; the execution of the module for identifying suspected prostate lesion areas comprising the processing of ADC maps and diffusion-weighted images (DWI) for the identification of suspected prostate lesion areas, each of the identified suspected areas having a centroid; and the execution of the module for classifying lesions which comprises a classifier that is fed by cubes of predetermined area centered on the centroids of the suspected prostate lesion areas, the classifier comprising a first classifier algorithm, which is fed with slices of the cubes and generates a probability of clinical significance of the lesion, and a second classifier algorithm, which is fed with the probability generated by the first algorithm, information from the module for zonal segmentation of the prostate, and statistical information obtained from the T2-weighted image sequences, to provide a probability of suspected areas of clinically significant cancer.

In one embodiment of the invention, the algorithm for segmenting the prostate peripheral and transitional zones is an algorithm trained with manual delimitation data of the prostate peripheral and transitional zones. Preferably, the algorithm for segmenting the prostate peripheral and transitional zones is an algorithm based on a convolutional neural network (CNN) based on the 2D U-Net topology.

T2-weighted image sequences fed into the module for zonal segmentation of the prostate can be previously processed with adaptive equalization, image normalization, and central cut.

In the module for identifying suspected prostate lesion areas, the processing of ADC maps and diffusion-weighted images (DWI) comprises:

a) the application of a ReLu filter for the identification of areas of congruence in the image, the ReLu filter being given by the difference between the ADC and DWI images, following the equation:

F(x,y,z)=max(0,ADC(x,y,z)−DWI(x,y,z))

b) application of an agglomerative clustering process for aggregation of voxels close to the identified areas of congruence; and

c) identification the suspected prostate lesion areas by combining the identified areas of congruence with the aggregated voxels.

The cubes of predetermined area centered on the centroids of the suspected prostate lesion areas are preferably cubes with 30 mm edges.

Preferably, the first classifier algorithm of the module for classifying lesions is a VGG-16 convolutional network modified in 2D and the second classifier algorithm is a random forest algorithm.

### BRIEF DESCRIPTION OF THE DRAWINGS

The present invention will be described in more detail below, with references to the accompanying drawings, in which:

FIG. 1—is a schematic flowchart of the method for identifying and classifying prostate lesions in multi-parametric magnetic resonance images in accordance with the present invention;

FIG. 2—is an illustration of the manual delimitation of the prostate transitional and peripheral zones in a magnetic resonance image;

FIG. 3—is a schematic flowchart of the zonal segmentation module of the method for identifying and classifying prostate lesions in multi-parametric magnetic resonance images in accordance with the present invention;

FIG. 4—is a schematic flowchart of the module for identifying suspected prostate lesion areas of the method for identifying and classifying prostate lesions in multi-parametric magnetic resonance images in accordance with the present invention;

FIG. 5—is a schematic flowchart of the prostate module for classifying lesions of the method for identifying and classifying prostate lesions in multi-parametric magnetic resonance images in accordance with the present invention;

FIG. 6—is an illustration of the segmentation metrics of the datasets resulting from the segmentation module evaluation, considering the delimitation of the prostate transitional zone;

FIG. 7—is an illustration of the segmentation metrics of the datasets resulting from the segmentation module evaluation, considering the delimitation of the prostate transitional zone, when related to the radiologists' notes;

FIG. 8—is an illustration of the segmentation metrics of the datasets resulting from the segmentation module evaluation, considering the delimitation of the prostate peripheral zone;

FIG. 9—is an illustration of the segmentation metrics of the datasets resulting from the segmentation module evaluation, considering the delimitation of the prostate peripheral zone, when related to the radiologists' notes;

FIG. 10—illustrates the cross-validation (CV) ROC curve of the classification algorithm evaluation considering a test dataset;

FIG. 11—illustrates the cross-validation (CV) ROC curve of the classification algorithm evaluation considering another test dataset;

FIG. 12—illustrates the logic of prostate segmentation of the peripheral region segmentation module using the segmentation information from the transitional and entire prostate models;

FIG. 13—illustrates the neural network topology used for the segmentation of the entire prostate, considering the left input (image);

FIG. 14—illustrates the neural network topology used for segmentation of the entire prostate, considering the right input (image);

FIG. 15—illustrates the neural network topology used for the segmentation of the entire prostate, considering the main input (image);

FIG. 16—illustrates the neural network topology used for the segmentation of the transitional region of the prostate, considering the left input (image);

FIG. 17—illustrates the neural network topology used for segmentation of the transitional region of the prostate, considering the right input (image);

FIG. 18—illustrates the neural network topology used for the segmentation of the transitional region of the prostate, considering the main input (image); and

FIG. 19—illustrates the neural network topology used to classify prostate lesions.

### DETAILED DESCRIPTION OF THE INVENTION

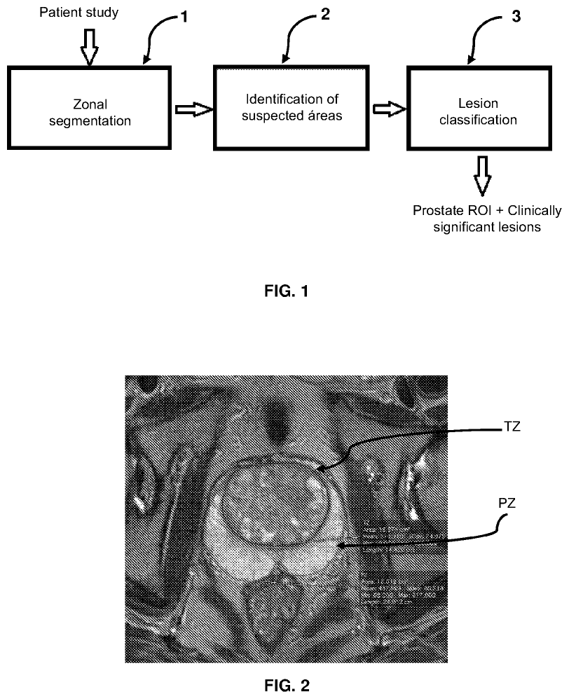

The present invention will be described below based on embodiments of the invention illustrated in FIGS. 1 to 19.

As illustrated in FIG. 1, the method for identifying and classifying prostate lesions in multi-parametric magnetic resonance images of the present invention comprises the execution of three modules: a module for zonal segmentation of the prostate (1), a module for identifying suspected areas (2), and a module for classifying lesions (3).

The module for zonal segmentation of the prostate comprises an algorithm for segmenting, from T2-weighted image sequences of multi-parametric magnetic resonance images, the prostate peripheral and transitional zones.

Preferably, the algorithm for segmenting the prostate peripheral and transitional zones is an algorithm trained with manual delimitation data of the prostate peripheral and transitional zones. Such manual delimitation can be done by an experienced professional, such as e.g., a radiologist with experience in multi-parametric magnetic resonance images.

FIG. 2 shows an example of manual delimitation, where the transitional zone (TZ) and the peripheral zone (PZ) can be seen.

The segmentation in transitional and peripheral zones makes the identification of clinically significant lesions more assertive, once studies show that 90% of malignant lesions are in the peripheral region. Thus, depending on the location, the analysis is performed differently.

Thus, in the method of the present invention, the zonal segmentation of the prostate is one of the inputs of the module for classifying lesions.

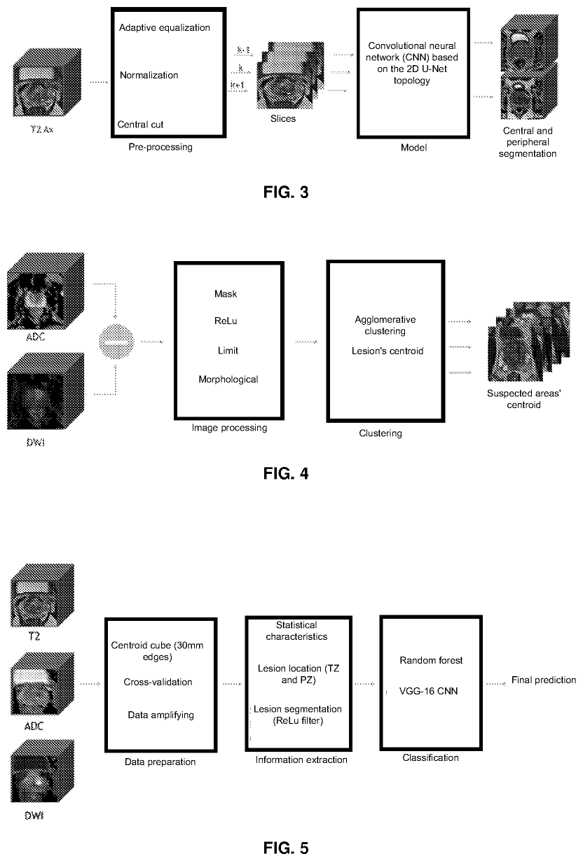

As better illustrated in the schematic flowchart of FIG. 3, in an embodiment of the invention, the zonal segmentation of the prostate uses an algorithm based on a convolutional neural network (CNN) based on the U-Net 2D topology to perform the segmentation of the entire prostate, delimiting the TZ and PZ, initially using T2-weighted axial series images.

Before performing the segmentation, algorithms can be applied to pre-process the images, including adaptive equalization, followed by image normalization and 80% central cut for TZ and 40% for PZ.

FIG. 4 schematically illustrates the execution of the module for identifying suspected prostate lesion areas (2).

Thus, the module for identifying suspected prostate lesion areas comprises the processing of ADC maps and diffusion-weighted images (DWI) to identify suspected prostate lesion areas, each of the identified suspected areas having a centroid.

Thus, the suspected areas identification algorithm of the second module applies image processing methods on ADC and DWI maps to locate diffusion-restricted areas. A combination of images is filtered by signal strengths and followed by morphological operations resulting in some sparse spots in the prostate.

These image processing methods can comprise the application of a ReLU filter by the difference between ADC and DWI images, following the equation:

F(x,y,z)=max(0,ADC(x,y,z)−DWI(x,y,z))

After applying the ReLU filter, processing can be performed to merge and fill the cluster of nearby voxels, and such voxels are later grouped through an agglomerative clustering process, so that closer voxels are considered as from the same suspected area for analysis.

Thus, the output of the module for identifying suspected prostate lesion areas (2) comprises suspected areas centroids.

The ReLU filter applied by the module for identifying suspected areas is based on the clinical observation that a radiologist makes to diagnose the image. As lesions glow on DWI (b-valued) images and are dark on ADC images, subtraction allows for the highlighting of zones that coincided positively, allowing the identification of areas of congruence.

FIG. 5 schematically illustrates the execution of the module for classifying lesions.

The classification module comprises a classifier that is powered by cubes of predetermined area centered on the centroids of the suspected prostate lesion areas. Preferably, the cubes centered on the centroids are cubes with 30 mm edges. This cube size value was chosen to ensure coverage of entire 15 mm lesions, even if the identified centroid is at the edge of the lesion.

The classifier comprises a first classifier algorithm, which is fed with cube slices and generates a probability of clinical significance of the lesion, and a second classifier algorithm, which is fed with the probability generated by the first algorithm, information from the module for zonal segmentation of the prostate and statistical information obtained from the T2-weighted image sequences, to provide a probability of suspected areas of clinically significant cancer.

Preferably, the first classifier algorithm of the module for classifying lesions is a VGG-16 convolutional network modified in 2D and the second classifier algorithm is a random forest algorithm.

It is important to highlight that the method of the present invention does not use K-trans type sequences. Thus, it is not necessary for the patient to ingest contrast to generate the images, which brings advantages associated with cost reduction, mitigation of allergy risks and pulmonary complications in patients with chronic kidney diseases.

### EXEMPLARY EMBODIMENT OF THE METHOD OF THE PRESENT INVENTION

Data Used in the Exemplary Embodiment of the Method of the Present Invention

For the amplifying embodiment of the method of the present invention, data from 163 anonymous patients randomly selected from patients undergoing both multi-parametric magnetic resonance imaging and subsequent biopsy or prostatectomy within a maximum interval of 6 months were used. The only inclusion criterion was the clinical indication for an mp-MRI, that is, a clinical suspicion of prostate cancer due to an increase in PSA levels and/or an alteration in the digital rectal examination. The only exclusion criteria were contraindications to the method, such as the use of devices not compatible with MRI or claustrophobia.

All images were acquired on three Tesla scanners without endorectal coil, following the standard mp-MRI protocol [information on the acquisition protocol can be found in the following references: “PI-RADS: Prostate Imaging—Reporting and Data System”-ACR-Radiology, 2015; Mussi, Thais Caldara et. al.; “Are Dynamic Contrast-Enhanced Images Necessary for Prostate Cancer Detection on multi-parametric Magnetic Resonance Imaging?”, Clinical Genitourinary Cancer, Volume 15, 3rd edition, e447-e454; Mariotti, G. C., Falsarella, P. M., Garcia, R. G. et. al. “Incremental diagnostic value of targeted biopsy using mpMRI-TRUS fusion versus 14-fragments prostatic biopsy: a prospective controlled study”. Eur Radiol (2018) 28:11. Available at https://doi.orq/10.1007/s00330-017-4939-01].

The image data sequences used to develop and train the method algorithms were: T2-weighted and diffusion-weighted (DWI) axial sequences, the last being with a B value of 800 and together with its post-processed ADC map.

To develop and test the ranking stage, it was also used an external dataset from the international competition PROSTATEx Challenge 2017 [available at Armato, Samuel G., Nicholas A. Petrick, and Karen Drukker. “PROSTATEx: Prostate MR Classification Challenge (Conference Presentation).” SPIE Proceedings, Volume 10134, id. 101344G 1 pp. (2017). 134 (2017)]. This dataset consists of 204 exams also acquired in 3T MRI without endorectal coil, but from multivendor machines. Of these 204 patients, the dataset provides 314 confirmed lesions annotated, 72 clinically significant and 242 non clinically significant.

Data Preparation

Each mp-MRI exam was initially prepared to create a benchmark dataset (ground truth) for the zonal segmentation tasks and the identification and classification of prostate lesions.

The first consisted of the zonal segmentation module. For this, all slices of the axial acquisitions in T2 of all exams included in the mp-MRI were analyzed individually and the zonal segmentation of the prostate was manually delimited showing the peripheral zone (PZ) and the transitional zone (TZ).

As shown in FIG. 2, manual delimitation of the peripheral and transitional zones was always performed and/or verified by an abdominal radiologist with more than two years of experience in multi-parametric magnetic resonance imaging. Among all exams, 19 were also verified by a second radiologist, with one year of experience in mp-MRI reading, in order to create a second specific dataset to assess inter-operator variability for prostate segmentation.

The second step consisted of creating a true reference dataset (ground truth) for the lesion detection and classification algorithm. To do this, 88 of the 163 series of images were used and classified following PI-RADS v2 guidelines [available at Weinreb, Jeffrey C., et al. “PI-RADS prostate imaging-reporting and data system: 2015, version 2.” European urology 69.1 (2016): 16-40]. Again, all these exams were analyzed individually by the same two-year experienced prostate radiologist, and 67 of them had no significant findings on MRI (PI-RADS 1-2) and a negative random biopsy. Thus, these 67 exams were considered as true negatives in the dataset. The other 21 scans had at least one indeterminate area or one suspected area of a clinically significant lesion on mp-MRI (PI-RADS 3 or 4-5, respectively), and this area was confirmed as a significant tumor (Gleason>6) in biopsy performed with mp-MRI-US combination or prostatectomy. Thus, these 21 were considered as true positives in the dataset. For these cases, all lesions were noted, indicating the lesion centroids in the 3D series.

Computer-Aided Blurring and Classification Method According to the Exemplary Embodiment of the Method of the Present Invention

The method of the present invention comprises three modules: (1) a zonal segmentation module, (2) a module for identifying suspected areas, and (3) a module for classifying lesions.

The zonal segmentation module comprises an algorithm based on a convolutional neural network (CNN) based on the U-Net 2D topology to perform the segmentation of the entire prostate, delimiting the transitional zone (TZ) and the peripheral zone (PZ).

An example of the topology used is that proposed by Ronneberger, Fischer and Brox, T. in the article “U-Net: Convolutional Networks for Biomedical Image Segmentation.”

For the zonal segmentation module, images from the T2-weighted axial series are initially used.

Before performing the segmentation, algorithms are applied to pre-process the images, including adaptive equalization, followed by image normalization and 80% central cut for TZ and 40% for PZ.

An example of adaptive equalization preprocessing is proposed by Pfizer et al. in the article “Adaptive histogram equalization for automatic contrast enhancement of medical images” [Pizer, Stephen M, et al. “Adaptive histogram equalization for automatic contrast enhancement of medical images.” Application of Optical Instrumentation in Medicine XIV and Picture Archiving and Communication Systems. Vol. 626. International Society for Optics and Photonics, 1986]. An example of image normalization is proposed by Hackeling in the article “Mastering Machine Learning with scikit-learn” [Hackeling, Gavin. Mastering Machine Learning with scikit-learn. Packt Publishing Ltd, 2017].

The segmentation algorithm was trained with 100 patients and validated in 44 patients, then the final model was chosen based on the best score obtained in the validation dataset during the training process.

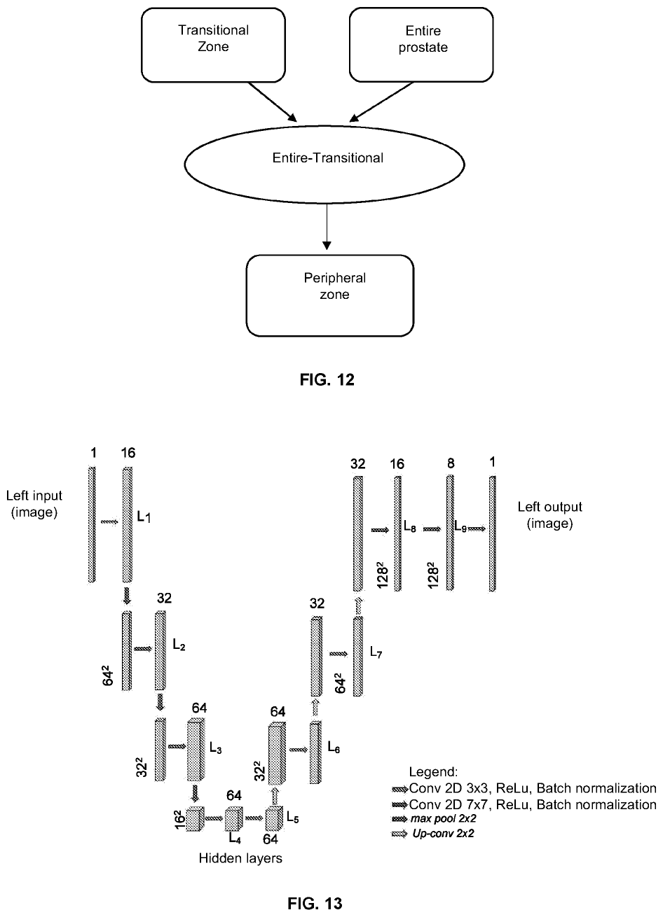

As illustrated in FIG. 12, the logic adopted for segmentation comprises the segmentation of the entire prostate and the segmentation of the transitional prostate. The peripheral region segmentation is basically the segmentation of the transitional region minus the entire prostate.

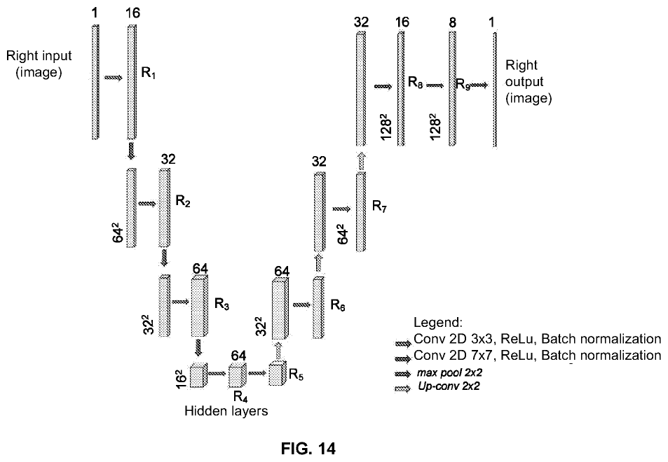

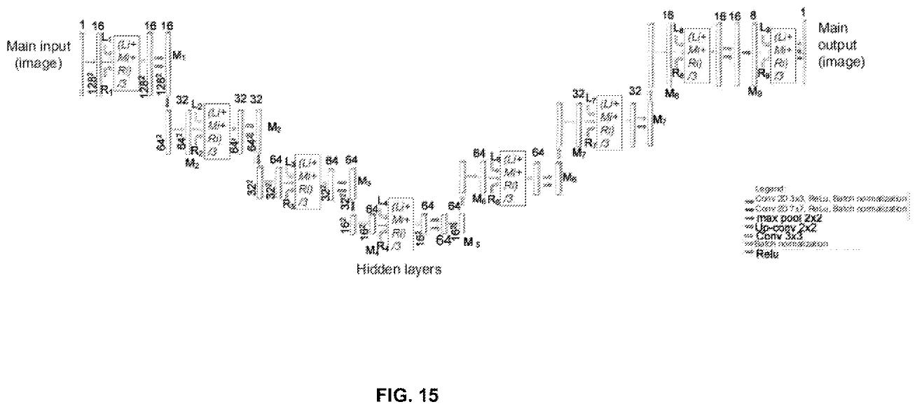

FIGS. 13 to 15 show the topology of the neural network used for segmentation of the entire prostate, with FIG. 13 being the left input (image), FIG. 14 the right input (image) and FIG. 15 the central input (image).

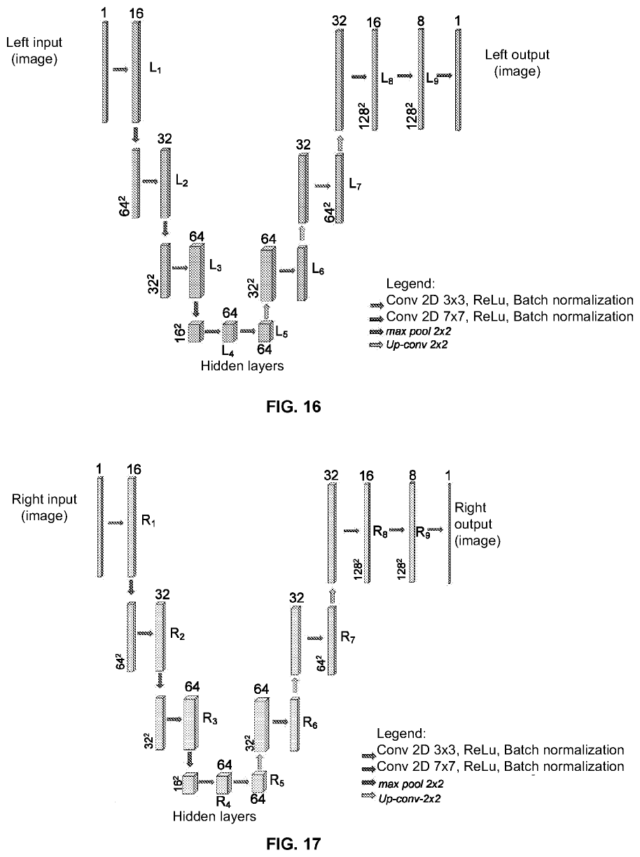

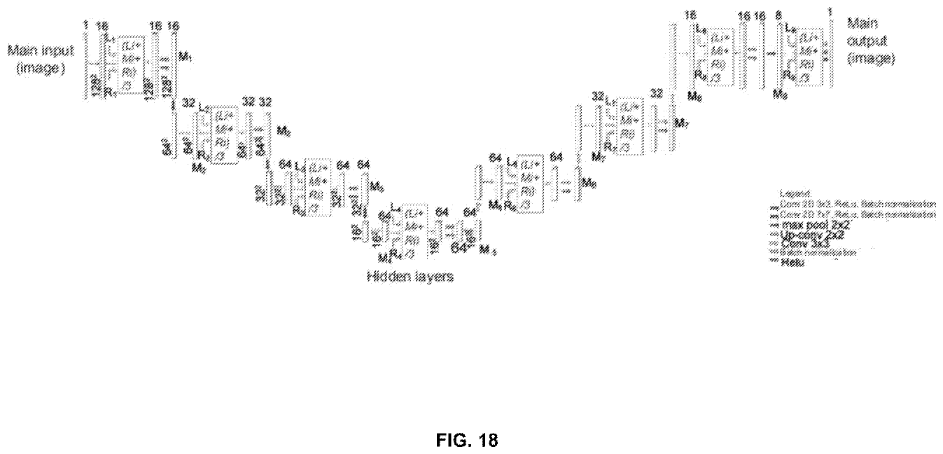

FIGS. 16 to 18 show the topology of the neural network used for the segmentation of the transitional zone, with FIG. 16 being the left input (image), FIG. 17 the right input (image) and FIG. 18 the central input (image).

The suspected areas identification algorithm of the second module applies image processing methods on ADC and DWI maps to locate diffusion-restricted areas. A combination of images is filtered by signal strengths and followed by morphological operations resulting in some sparse spots in the prostate.

These image processing methods comprise the application of a ReLU filter by the difference between ADC and DWI images, following the equation:

F(x,y,z)=max(0,ADC(x,y,z)−DWI(x,y,z))

After that, an opening and a closing operation is applied to merge and fill the cluster of nearby voxels. An example of this type of operation is proposed by Gonzales and Woods in the article “Digital Image Processing” [Gonzalez, Rafael C., and Richard E. Woods. “Image Processing.” Digital image Processing 2 (2007)].

These voxels are then grouped through an agglomerative clustering process, so that closer voxels are considered as from the same suspected area for analysis. An example of an agglomerative clustering process is proposed by Duda and Hart in the article “Pattern classification and scene analysis.” [Dudley, Richard O., and Peter E. Hart. “Pattern classification and scene analysis.” A Wiley-Interscience Publication, N.Y.: Wiley, 1973 (1973)].

The last module of the method comprises the lesion classification algorithm. This algorithm receives the centroids of these suspected areas to classify them according to clinical significance.

The classifier was developed as a combination of two models whose inputs are 30 mm cubes in the centroid of suspected areas. This cube size value was chosen because the P1-RADS 5 cutoff (the highest note) is of 15 mm. Thus, the choice guarantees the coverage of hole lesions with 15 mm, even if the identified centroid is at the edge of the lesion.

The image sequences used for this step were: T2-weighted axial, DWI and ADC map.

The first classifier model comprises a modified 2D VGG-16 convolutional network that receives the cube slices and generates the probability of clinical significance. An example of VGG-16 is proposed by Simonyan and Zisserman [Simonyan, K., & Zisserman, A. (2014). Very Deep Convolutional Networks for Large-Scale Image Recognition].

The second classifier model is a random forest classifier that combines the VGG outputs with statistical characteristics (Maximum, Mean, Standard Deviation, Asymmetry and Kurtosis), in addition to the tumor location (TZ or PZ) obtained in the segmentation step. The final result is the probability of clinically significant cancer suspected areas.

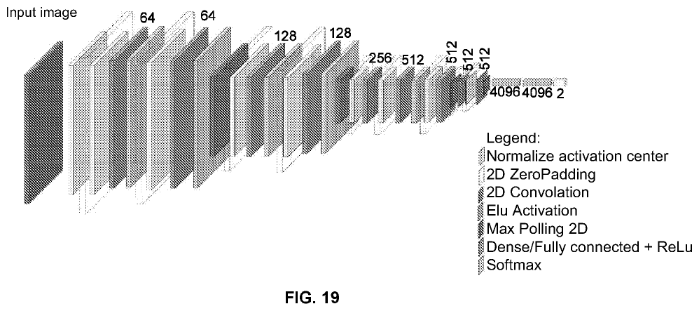

FIG. 19 shows the prostate lesion classification network topology.

Specifically for this classification step, the model training and validation process used the external dataset of the PROSTATEx Challenge 2017 international contest.

Statistical Evaluation of the Exemplary Embodiment of the Method of the Present Invention

Statistical evaluation for each module of the method of the present invention was performed with the aim of judging each module as a different part of the method.

Segmentation Module

First, for the segmentation module, the DICE coefficient, the sensitivity and the Hausdorff 95 distance were considered as evaluation metrics.

The DICE coefficient (equation 1), also called the overlap index, is the most used metric in validating medical volume segmentations. It performs a harmonic average between what was predicted (X) and the fundamental truth (Y):

$$DSC = \frac{2 \vert X ⋂ Y \vert}{\vert X \vert + \vert Y \vert} \qquad Equation 1$$

Sensitivity, Recall or true positive rate (TPR), measures the portion of positive voxels in the ground truth (TP) that are also identified as positive by the segmentation being evaluated (TP+FN), as described in equation 2:

$$Sensitivity = TPR = \frac{TP}{TP + FN} \qquad Equation 2$$

Considering that the segmentation output will be used to look for suspected areas in the prostate (second module), it is desirable that the entire gland is analyzed, which means that sensitivity assessment is important.

And finally, completing the analysis with a spatial distance metric, the Hausdorff distance (HD) was also considered.

As HD is generally sensitive to outliers, which is quite common in medical image segmentations, we considered the HD quantile to better assess the spatial positions of the voxels.

Considering A, B two subsets of non-empty and finite points, the Hausdorff directed distance h (A, B) corresponds to the value of the maximum distance of the set with normal values ∥a-b ∥, for example, Euclidean distance. HD (A, B) is obtained by the maximum value of h, as shown in the next equations:

$$h ( A , B ) = \begin{matrix}max \\ a ∈ A\end{matrix} \begin{matrix}min \\ b ∈ B\end{matrix} ‖ a - b ‖ \\ HD ( A , B ) = max ( h ( A , B ) , h ( B , A ) ) \qquad Equations 3 and 4$$

Module for Identifying Suspected Areas

Second, the sensitivity score was also used to assess the identification of suspected areas. This metric was considered sufficient, as the objective of this module is to avoid the loss of true regions with lesions, that is, false negatives (FN) are undesirable and false positives (FR) are indifferent because the responsibility for eliminating them belongs to the classifier in the next step. To correctly apply the sensitivity in the lesion detection problem, a maximum distance of 5 mm between the identified centroids (by the algorithm) and the target (reference value—ground truth) was considered as a criterion for representing the same area.

Module for Classifying Lesions

Finally, the module for classifying lesions was evaluated using the Receiver Operating Characteristic (ROC) curve and its area under the curve. It has a significant interpretation for the classification of diseases of healthy individuals and was also adopted as the classification metric by the PROSTATEx Challenge 2017, making it possible to better compare the method's performance with the state of the art for classification of prostate lesions.

Experimental Results of the Evaluation of the Exemplary Embodiment of the Method of the Present Invention

Zonal Segmentation

Considering the database of 163 patients for the zonal segmentation step, 44 patients were selected as the validation dataset and 19 patients were selected as the test dataset.

One of the 44 validation cases was excluded from the set, as its multi-parametric resonance was acquired after a prostatectomy procedure.

Transitional Zone (TZ) Segmentation

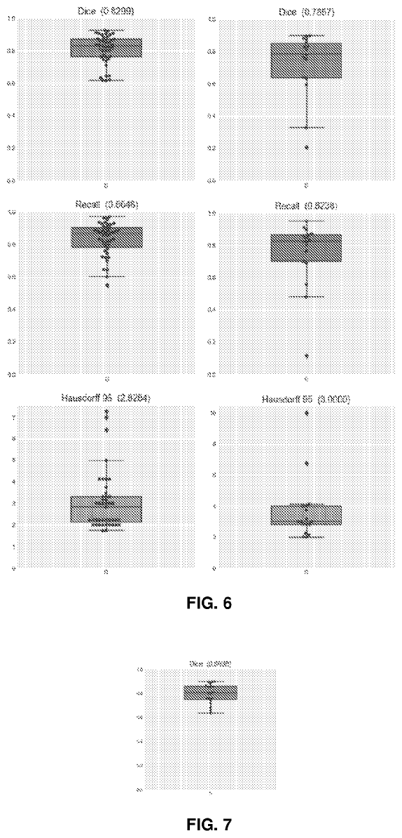

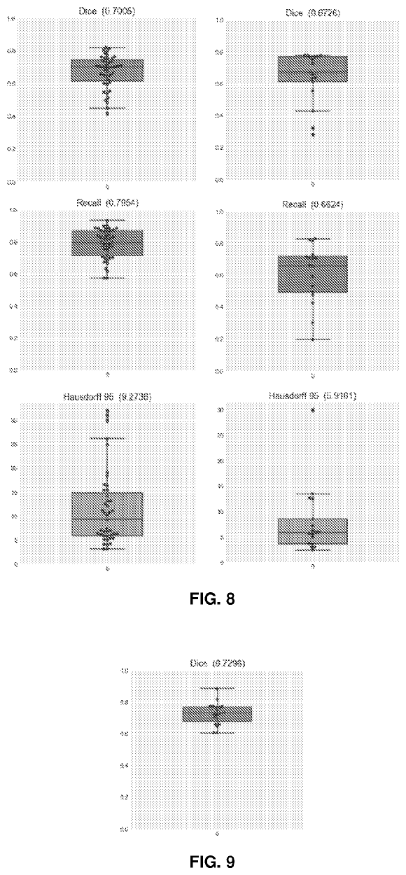

Table 1 below and the distributions shown in FIG. 6 present the segmentation metrics of the datasets. In addition, FIG. 7 shows the DICE distribution between the field notes of two radiologists, in order to illustrate the inter-operator variability of the problem.

TABLE 1
Summary of transitional zone
segmentation metrics

| | | | Hausdorff |
|---|---|---|---|
| | Dice | Recall | 95 Distance |
| TZ | (average) | (average) | (average) |
| Validation | 0.8299 | 0.8646 | 2.8284 |
| Test | 0.7857 | 0.8238 | 3.0000 |

The average DICE scores obtained were 0.8083 between the two radiologists' notes and 0.7857 between the algorithm and the most experienced radiologist's note, with a difference of 0.8038-0.7857=0.0181 (relative error=0.025).

Peripheral (PZ) Zone Segmentation

Analogous to TZ segmentation, table 2 below and the distributions shown in FIG. 8 present the segmentation metrics of the datasets. In addition, FIG. 9 shows the DICE distribution between the field notes of two radiologists, in order to illustrate the inter-operator variability of the problem.

TABLE 2
Summary of peripheral zone
segmentation metrics

| | | | Hausdorff 95 |
|---|---|---|---|
| | Dice | Recall | Distance |
| PZ | (average) | (average) | (average) |
| Validation | 0.7005 | 0.7954 | 9.2736 |
| Test | 0.6726 | 0.6624 | 5.9161 |

The PZ segmentation evaluation presented a similar behavior to the TZ segmentation evaluation, with DICE values relatively close between the test and the radiologists, with a relative error of 0.0781.

The result of the interoperator analysis for PZ and TZ segmentation makes it possible to quantify radiologists' conformity for the test dataset. Although only two observer radiologists were used, the average data between the radiologists was similar to the data between the algorithm and the radiologist, which demonstrates that the algorithm was able to perform an analysis similar to the radiologists' one.

Module for Identifying Suspected Areas

For the experiment of the module for identifying suspected areas, 21 patients out of a total of 88 patients were evaluated, with 22 confirmed lesions. Two of the 22 confirmed lesions were located in the seminal vesicle and were excluded from the identification analysis for this reason, resulting in 20 lesions to be identified as suspected areas.

The sensitivity of the suspected areas identification step was of 1.0 (100%), detecting all 20 lesions considered. Its rate of findings was of 1.85 suspected areas per true lesion, out of a total of 37.

Thus, the results demonstrate that the module for identifying suspected areas is able to automate the search for clinically significant lesions by tracking diffusion restriction areas with image processing methods.

Classification of Prostate Lesions

For the evaluation of the module for classifying lesions, two different datasets were used: a set with 88 exams and a set with 204 exams (PROSTATEx).

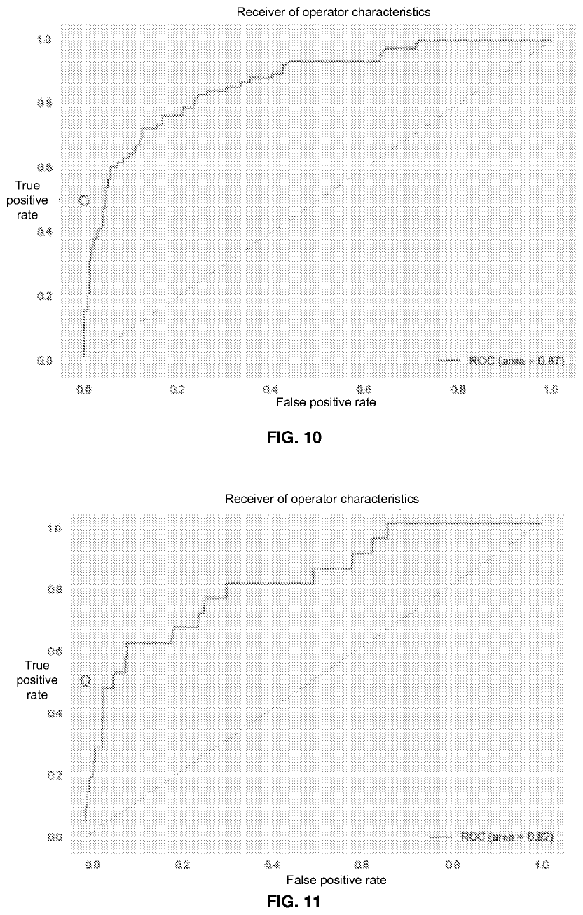

As shown in FIG. 10, the 204 PROSTATEx series, with 314 clinically significant and non-clinically significant lesions, was used by cross validation (CV), using 5 partitions to assess the performance of the algorithm. The area below the ROC Curve corresponds to the competition CV score.

The 5-partition cross-validation confusion matrix applied to the PROSTATEx training dataset is shown in table 3 below.

TABLE 3
Confusion matrix

| | | Predicted | |
|---|---|---|---|
| | | Label | |
| | | False | True |
| True Label | False | 190 | 52 |
| | True | 22 | 50 |

Table 4 below shows the Precision and Recall metrics for the 5-partition cross-validation applied to the PROSTATEx training dataset.

TABLE 4
Metrics (CV)

| Class | Precision | Recall |
|---|---|---|
| False | 0.90 | 0.79 |
| True | 0.49 | 0.69 |

The classification module algorithm was also evaluated on the 88-exam test database to make a more robust analysis regarding the generalizability of the model and to assess its performance without K-trans maps. The AUCROC obtained was 0.82 for the test dataset (see FIG. 11).

Having described examples of embodiments of the present invention, it should be understood that the scope of the present invention encompasses other possible variations of the described inventive concept, being limited only by the content of the appended claims, including possible equivalents therein.

1. A method for identifying and classifying prostate lesions in multi-parametric magnetic resonance images, comprising:
executing a module for zonal segmentation of a prostate comprising an algorithm for segmenting, from T2-weighted image sequences of the multi-parametric magnetic resonance images, prostate peripheral and transitional zones;
executing a module for identifying suspected prostate lesion areas comprising processing ADC maps and diffusion-weighted images (DWI) to identify suspected prostate lesion areas, each of the identified suspected areas having a centroid; and
executing a module for classifying lesions comprising a classifier that is fed by cubes of predetermined area in the centroids of the identified suspected prostate lesion areas, the classifier comprising a first classifier algorithm, which is fed with cube slices and generates a probability of clinical significance of the lesions, and a second classifier algorithm, which is fed with the probability generated by the first algorithm, information from the module for zonal segmentation of the prostate and statistical information obtained from the T2-weighted image sequences, to provide a probability of suspected areas of clinically significant cancer.

2. The method according to claim 1, wherein the algorithm for segmenting the prostate peripheral and transitional zones is an algorithm trained with manual delimitation data of the prostate peripheral and transitional zones.

3. The method according to claim 2, wherein the algorithm for segmenting the prostate peripheral and transitional zones is an algorithm based on a convolutional neural network (CNN) based on 2D U-Net topology.

4. The method according to claim 3, wherein the T2-weighted image sequences fed into the module for zonal segmentation of the prostate are previously processed with adaptive equalization, image normalization, and central cut.

5. The method according to claim 1, wherein the processing of ADC maps and diffusion-weighted images (DWI) of the module for identifying suspected prostate lesion areas comprises:
a) applying a ReLu filter for identification of areas of congruence in the images, the ReLu filter being given by the difference between the ADC and DWI images, following the equation:
F(x,y,z)=max(0,ADC(x,y,z)−DWI(x,y,z));
b) applying an agglomerative clustering process for aggregation of voxels close to the identified areas of congruence; and
c) identifying the suspected prostate lesion areas by combining the identified areas of congruence with the aggregated voxels.

6. The method according to claim 1, wherein the cubes of predetermined area centered on the centroids of the suspected prostate lesion areas are cubes with 30 mm edges.

7. The method according to claim 1, wherein the first classifier algorithm of the module for classifying lesions is a VGG-16 convolutional network modified in 2D and the second classifier algorithm is a random forest algorithm.
